(19) United States
(12) Patent Application Publication
(10) Pub. No.: US 2023/0083072 A1
(43) Pub. Date: Mar. 16, 2023

(54) FILM-COATED GRANULE, PHARMACEUTICAL PREPARATION CONTAINING THE SAME, AND MANUFACTURING METHODS THEREOF

(71) Applicant: SAWAI PHARMACEUTICAL Co., Ltd., Osaka (JP)

(72) Inventors: Katsuhiko OMAGARI, Osaka (JP); Shinya TAKADA, Osaka (JP); Ryota KIMATA, Osaka (JP); Naoki YOSHIHARA, Osaka (JP)

(21) Appl. No.: 17/941,129

(22) Filed: Sep. 9, 2022

(30) Foreign Application Priority Data
Sep. 10, 2021 (JP) 2021-148154
Aug. 19, 2022 (JP) 2022-131099

(51) Int. Cl. A61K 9/16; A61K 45/06

(52) U.S. Cl. CPC A61K 9/167; A61K 9/1652; A61K 9/1635; A61K 45/06; A61K 9/1682; A61K 9/1611; A61K 9/1617

(57) ABSTRACT

One of the problems of one embodiment of the present invention is to provide film-coated granules which have a new film constitution. Alternatively, one of the problems of one embodiment of the present invention is to provide a pharmaceutical preparation containing the film-coated granules which have a new film constitution. Alternatively, one of the problems of one embodiment of the present invention is to provide a new dry manufacturing method of the film-coated granules. Alternatively, one of the problems of one embodiment of the present invention is to provide a new dry manufacturing method of the pharmaceutical preparation containing the film-coated granules. According to one embodiment of the present invention, a film-coated granule is provided that comprises a core particle having a melt component, and a film arranged on a surface of the core particle, wherein the film includes a porous substance, a plasticizer and a polymer.

### CROSS-REFERENCE TO RELATED APPLICATIONS

This application claims the benefit of priority to Japanese Patent Application No. 2021-148154, filed on Sep. 10, 2021, and Japanese Patent Application No. 2022-131099, filed on Aug. 19, 2022, the entire contents of which are incorporated herein by reference.

### FIELD

The present invention relates to film-coated granules. Alternatively, the present invention relates to a pharmaceutical preparation containing the film-coated granules. Alternatively, the present invention relates to a manufacturing method of the film-coated granules. Alternatively, the present invention relates to a manufacturing method of the pharmaceutical preparation containing the film-coated granules.

### BACKGROUND

In pharmaceutical preparations, a coating is performed in various uses such as a dissolution control of an active ingredient such as gastric, enteric and sustained releases and masking of a bitter taste of an active ingredient. Coating methods are roughly divided into dry methods and wet methods. Wet methods generally require extensive manufacturing time for spraying of a solvent and drying. A solvent may adversely affect stability of the active ingredient in wet methods. For these reasons, for example, in Japanese patent publication No. 3417772, a method in which while a mixture of a liquid substance having a contact angle of 10° or less with respect to a polymer coating agent and a plasticizer is continuously sprayed onto the solid drug, the powdery polymer coating agent is spray coated onto the solid drug is described as a method which does not use a solvent. However, according to the method descried in the Japanese patent publication No. 3417772, special equipment is required so that a fixed amount of powder can be fed into the production equipment. In addition, the granules tend to aggregate in the method descried in the Japanese patent publication No. 3417772 and the method can only be applied to large particles and tablets.

On the other hand, in Japanese patent publication No. 6067154, a method is described for crushing particles after a layering step of attaching a polymer to the particles having a core and a wax layer over it in a dry process. However, because many of existing technologies in the dry process are coatings in a powdery state, formed film has no compactness and may not obtain the desired function. In addition, when compact film is formed by the existing technologies, there is a problem that a polymer used for coating is limited to a polymer having a low minimum film forming temperature.

### SUMMARY

One of the problems of one embodiment of the present invention is to provide film-coated granules which have a new film constitution. Alternatively, one of the problems of one embodiment of the present invention is to provide a pharmaceutical preparation containing the film-coated granules which have a new film constitution. Alternatively, one of the problems of one embodiment of the present invention is to provide a new dry manufacturing method of the film-coated granules. Alternatively, one of the problems of one embodiment of the present invention is to provide a new dry manufacturing method of the pharmaceutical preparation containing the film-coated granules.

According to one embodiment of the present invention, a film-coated granule is provided that comprises a core particle having a melt component, and a film arranged on a surface of the core particle, wherein the film includes a porous substance, a plasticizer and a polymer.

The plasticizer may be selected from plasticizers arrangeable between molecules of the polymer.

The plasticizer may be one or more selected from a group consisting of triethyl citrate, polyethylene glycol, propylene glycol, triacetin and tributyl acetylcitrate, and the polymer may be one or more selected from a group consisting of hypromellose acetate succinate, hydroxypropyl cellulose, ethyl cellulose, vinyl acetate, methacrylic acid copolymer L, ammonioalkyl methacrylate copolymer and methacrylic acid copolymer S.

The core particle may include a core substance, a molten component layer arranged on a surface of the core substance, and an active ingredient-containing layer arranged on a surface of the molten component layer.

The core particle may include an active ingredient and the melt component, and the active ingredient and the melt component may be bound.

The core particle further may include a polymer, and the active ingredient, the melt component and the polymer may be bound.

The film may further include one or more first pharmaceutically acceptable additive agents.

According to one embodiment of the present invention, a pharmaceutical preparation is provided that comprises any one of the film-coated granules, and one or more second pharmaceutically acceptable additive agents.

According to one embodiment of the present invention, a manufacturing method of a film-coated granule is provided comprising adsorbing a plasticizer to a porous substance, adsorbing the porous substance absorbed with the plasticizer to a core particle having a melt component to obtain a first particle; and adsorbing a polymer to the first particle to form a film including the porous substance, the plasticizer, and the polymer on the core particle.

The polymer may be adsorbed to the first particle at a first temperature, and the film may be formed on the core particle at a second temperature equal to or higher than the first temperature.

The plasticizer may be selected from plasticizers arrangeable between molecules of the polymer.

The plasticizer may be one or more selected from a group consisting of triethyl citrate, polyethylene glycol, propylene glycol, triacetin and tributyl acetylcitrate, and the polymer may be one or more selected from a group consisting of hypromellose acetate succinate, hydroxypropyl cellulose, ethyl cellulose, vinyl acetate, methacrylic acid copolymer L, ammonioalkyl methacrylate copolymer and methacrylic acid copolymer S.

The melt component may be absorbed to a core substance to form a molten component layer, an active ingredient may be absorbed to the melt component, and an active ingredient-containing layer including the melt component and the active ingredient may be formed to obtain the core particle.

The melt component and an active ingredient may be bound, and the core particle including the melt component and the active ingredient may be obtained.

The melt component, an active ingredient and a polymer may be bound, and the core particle including the melt component, the active ingredient and the polymer may be obtained.

One or more pharmaceutically acceptable lubricants may be further added when forming the film on the core particle.

According to one embodiment of the present invention, a manufacturing method of a pharmaceutical preparation is provided that comprises mixing the film-coated granule obtained by any one of the above-described manufacturing method of the film-coated granule and one or more second pharmaceutically acceptable additive agents to obtain a mixture, and tableting the mixture.

### BRIEF DESCRIPTION OF DRAWINGS

FIG. 1 is a schematic diagram (cross-sectional view) which shows a film-coated granule 100 according to one embodiment of the invention.

FIG. 2 is a schematic diagram (cross-sectional view) which shows a core particle 10 according to one embodiment.

FIG. 3 is a flow diagram which illustrates a manufacturing method of a core particle 10 according to one embodiment of the invention.

FIG. 4 is a schematic diagram (cross-sectional view) which shows a core particle 20 according to one embodiment.

FIG. 5 is a flow diagram which illustrates a manufacturing method of a core particle 20 according to one embodiment.

FIG. 6 is a schematic diagram (cross-sectional view) which shows a film-coated granule 200 according to one embodiment.

FIG. 7A is a flow diagram which illustrates a step of preparing a porous substance 150 absorbing a plasticizer according to one embodiment.

FIG. 7B is a flow diagram which illustrates a manufacturing method of a film-coated granule 100 according to one embodiment.

FIG. 8A is a scanning electron microscope image of a granule of Example 1 before curing.

FIG. 8B is a scanning electron microscope image of a granule of Example 1 after curing.

FIG. 8C is a scanning electron microscope image of a granule of Example 2 before curing.

FIG. 8D is a scanning electron microscope image of a granule of Example 2 after curing.

FIG. 9A is a scanning electron microscope image of a granule of Comparative example 2 before curing.

FIG. 9B is a scanning electron microscope image of a granule of Comparative example 2 after curing.

FIG. 10 is a diagram showing elution ratios of duloxetine hydrochloride in film-coated granules of Example 3 and Comparative example 4.

### DESCRIPTION OF EMBODIMENTS

Hereinafter, a film-coated granule according to the present invention and a preparation including the same, and manufacturing methods thereof will be described with reference to drawings. In addition, a film-coated granule according to the present invention and a preparation including the same, and manufacturing methods thereof are not construed as being limited to the description contents of the embodiments and examples shown below. In the drawings referred to in the present embodiment and the examples described later, the same parts or parts having the same functions are designated by the same reference numerals, and the repeated description thereof will be omitted.

As described above, when the dry process is used, the compactness of the film is low due to attachment of the polymer in a powdery state. When a compact film is formed in the dry process, it is necessary to form a film by heating up to a temperature equal to or more than a minimum film forming temperature of the attached polymer to be softened. However, in view of a stability of an active ingredient to heat and a heat limitation of the manufacturing apparatus, a usable polymer is limited to a polymer having a low minimum film forming temperature. For this reason, when a polymer other than such a polymer is used, lowering of the minimum film forming temperature of the polymer is required. For example, in a wet process, a technique to lower the minimum film forming temperature of the polymer by addition of a plasticizer which lowers a glass transition point of the polymer is known. It is also expected that the minimum film forming temperature of the polymer is lowered by addition of a plasticizer in the dry process. On the other hand, an aggregation occurs by adhesion of particles to each other due to a fast reaction of the polymer with the plasticizer.

In the present invention, by use of a porous substance absorbing the plasticizer, the plasticizer gradually seeps from pores of the porous substance. Therefore, a rapid softening of the polymer caused by the fast reaction of the polymer with the plasticizer is avoided, then the above problem is solved.

### [Film-Coated Granules]

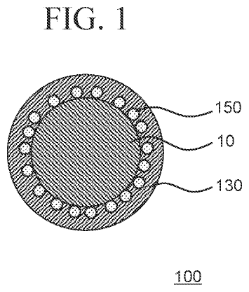

FIG. 1 is a schematic diagram (cross-sectional view) which shows a film-coated granule 100 according to one embodiment of the invention. The film-coated granule 100 includes a core particle 10 having a melt component, and a film 130 arranged on the core particle 10. The film 130 includes a porous substance 150, a plasticizer and a polymer. Although a manufacturing method of the film-coated granule 100 will be explained later, in the film-coated granule 100 according to the embodiment, the plasticizer absorbed on the porous substance 150 gradually seeps from the porous substance 150, and is mixed with the polymer, and the glass transition point of the polymer is lowered, when the film 130 is formed. Thereby, the film 130 becomes a compact film.

In the embodiment, it is not required that all the plasticizer seeps from the porous substance 150, and a part of the plasticizer may remain to be absorbed on the porous substance 150. The porous substance 150 can be selected from pharmaceutically acceptable porous substances. In one embodiment, “porous substance” means a particle in which numerous pores are arranged on a surface of the particle. In one embodiment, the porous substance 150 is a particle which meets at least one of 2 ml/g to 18 ml/g of bulk density, 110 m²/g to 700 m²/g of BET specific surface area, 0.4 cm³/g to 2.1 cm³/g of pore volume and 1 ml/g to 4 ml/g of oil absorption. In one embodiment, the porous substance 150 is a pharmaceutically acceptable porous silicate, and can be selected from hydrous silicon dioxide, light anhydrous silicic acid or magnesium aluminometasilicate. In one embodiment, the plasticizer is absorbed on the surface of the porous substance 150 and/or in the interior of the pores. In one embodiment, the film-coated granule 100 may contain 1 weight % to 30 weight % of the porous substance 150 with respect to 100 weight % of the film-coated granule.

In one embodiment, the plasticizer can be selected from a plasticizer arrangeable between molecules of the polymer. The plasticizer is selected from a group consisting of, for example, triethyl citrate, polyethylene glycol, propylene glycol, triacetin and tributyl acetylcitrate, but is not limited thereto. In one embodiment, the film-coated granule 100 may contain 1 weight % to 30 weight of the plasticizer with respect to 100 weight % of the film-coated granule.

In one embodiment, the polymer is selected from additive agents which can form the film, for example, a group consisting of hypromellose acetate succinate, hydroxypropyl cellulose, ethyl cellulose, vinyl acetate, methacrylic acid copolymer L, ammonioalkyl methacrylate copolymer and methacrylic acid copolymer S, but is not limited thereto. In one embodiment, the film-coated granule 100 may contain 1 weight % to 30 weight % of the polymer with respect to 100 weight % of the film-coated granule.

In one embodiment, the film may further include one or more pharmaceutically acceptable additive agents (first additive agents). Also, a constitution of the core particle 10 and porous substance 150 may be the same as the constitution described above and, a detailed explanation is omitted. Further, the polymer contained in the film 130 can be selected from the polymers described above. An additive agent capable of being contained in the film 130 is one or more additive agents selected from the group consisting of, for example, hypromellose, polyvinylpyrrolidone, polyvinyl alcohol, mannitol, erythritol, trehalose, lactose hydrate, crospovidone, starch, low-substituted hydroxypropylcellulose, talc, titanium oxide, stearic acid, magnesium stearate, light anhydrous silicic acid, hydrous dioxide silicon, dibutylhydroxytoluene, aspartame and sucralose.

### [Core Particle]

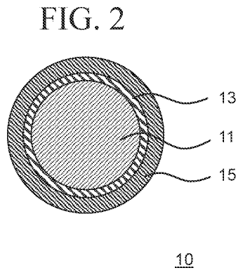

In the embodiment, the core particle 10 contains a melt component on at least its surface. Also, the core particle 10 contains an active ingredient. FIG. 2 is a schematic diagram (cross-sectional view) which shows a core particle 10 according to one embodiment. In the embodiment, the core particle 10 includes a core substance 11, a molten component layer 13 arranged on a surface of the core substance 11, and an active ingredient-containing layer 15 arranged on a surface of the molten component layer 13.

The core substance 11 is a support to arrange the molten component layer 13 and the active ingredient-containing layer 15, and a substance to be a core to arrange the molten component layer 13 and the active ingredient-containing layer 15 when the core particle 10 is manufactured. The core substance 11 can be selected from the group consisting of, for example, amberlite IRP-64, ion exchange resin, kaolin, carmellose calcium, hydrous silicon dioxide, magnesium silicate, light anhydrous silicic acid, light liquid paraffin, diatomaceous earth, synthetic aluminum silicate, aluminum oxide, aluminum hydroxide, absorbent cotton, magnesium carbonate, precipitated calcium carbonate, dextrin, silicon dioxide, composite potassium aluminum silicate grains, bentonite, polyethylene fiber, magnesium aluminometasilicate, medicinal charcoal, calcium silicate, cellulose acetate, anhydrous calcium hydrogen phosphate, microcrystalline cellulose, mannitol, sucrose, starch, lactose hydrate, and ammonioalkyl methacrylate copolymer. However, as described later, the core substance 11 is preferably an adsorbent such as amberlite IRP-64, ion exchange resin, kaolin, carmellose calcium, hydrous silicon dioxide, magnesium silicate, light anhydrous silicic acid, light liquid paraffin, diatomaceous earth, synthetic aluminum silicate, aluminum oxide, aluminum hydroxide, absorbent cotton, magnesium carbonate, precipitated calcium carbonate, dextrin, silicon dioxide, composite potassium aluminum silicate granules, bentonite, polyethylene fibers, magnesium aluminometasilicate, and medicinal charcoal.

The core substance 11 is preferably a spherical form to uniformly arrange the molten component layer 13 and the active ingredient-containing layer 15.

The molten component layer 13 is a layer arranged between the core substance 11 and the active ingredient-containing layer 15. The molten component layer 13 is an underlayer to arrange the active ingredient-containing layer 15. In the core particle 10, more active ingredients can be attached by arranging the molten component layer 13 on the surface of the core substance 11, thereby, a content of the active ingredient in the core particle 10 can be effectively increased.

“Melt component” means a component which can be melted by heat. A melt component constituting the molten component layer 13 is selected from oily additive agents. The melt component is selected from additive agents which are solid at room temperature to form the molten component layer 13 by a melt layering method. Considering a generally used temperature range in the melt layering, the melt component is preferably selected from additive agents which have a melting point equal to or lower than 100° C., and preferably selected from additive agents which have a melting point in a temperature range in which denaturation of the active ingredient or a significant increase of related substances is not recognized. As additive agents having such properties, glycerin monostearate, macrogol (polyethylene glycol), lauromacrogol, stearic acid, and the like can be exemplified, but the additive agents are not limited thereto. In addition, the melt component is preferably selected from additive agents which do not denature the active ingredient or show a significant increase in related substances due to contact with the active ingredient.

The molten component layer 13 may be arranged on the surface of the core substance 11 in an amount in which the active ingredient-containing layer 15 can be arranged, and may be arranged on at least a part of the surface of the core substance 11. The molten component layer 13 preferably covers 90% or more of the surface of the core substance 11, and preferably covers the entire surface of the core substance 11. The thickness of the molten component layer 13 is not particularly limited, but it is preferable that the thickness of the molten component layer 13 is as thin as possible from the viewpoint of increasing the active ingredient content per core particle 10. In one embodiment, it is preferable that the melt component constituting the molten component layer 13 is also arranged in pores of the surface of the core substance 11. In one embodiment, when the core substance 11 is an adsorbent, the interface between the core substance 11 and the molten component layer 13 may have a structure in which the melt component constituting the molten component layer 13 enters from the surface of the core substance 11. In this case, the core substance 11 and the molten component layer 13 do not have to have a clear interface. By arranging the melt component not only on the surface of the core substance 11 but also in the pores connected to the surface of the core substance 11, the molten component layer 13 is imparted with an anchor effect on the core substance 11 and the adhesion of the molten component layer 13 on the core substance 11 is improved.

The active ingredient-containing layer 15 is a layer containing at least the active ingredient, and is arranged on a surface of the molten component layer 13.

The active ingredient-containing layer 15 may further contain a melt component or polymer. In the case where an additive agent having a melting point lower than a melting point of the melt component is selected as the melt component contained in the active ingredient-containing layer 15, when the active ingredient-containing layer 15 is formed by the melt layering method, the active ingredient-containing layer 15 can be arranged on the surface of the molten component layer 13 without significantly affecting the surface structure of the molten component layer 13 or changing the surface structure of the molten component layer 13. On the other hand, in the case where an additive agent having a melting point higher than a melting point of the melt component is selected as the melt component contained in the active ingredient-containing layer 15, when the active ingredient-containing layer 15 is formed by the melt layering method, the surface of the molten component layer 13 is slightly melted, and the interface between the molten component layer 13 and the active ingredient-containing layer 15 is fused, so that the adhesion of the active ingredient-containing layer 15 to the molten component layer 13 can be improved.

As the additive agents used as the melt component contained in the active ingredient-containing layer 15, stearic acid, glycerin monostearate, macrogol (polyethylene glycol), carnauba wax, hardened oil, lauromacrogol, palmitic acid, cetyl alcohol and the like can be exemplified, but the additive agents are not limited thereto. The melt component contained in the active ingredient-containing layer 15 is preferably selected from additive agents which do not denature the active ingredient or show a significant increase in related substances due to contact with the active ingredient. From the viewpoint of adhering to the core substance 11, the particle size of the melt component needs to be smaller than the particle size of the core substance 11. Further, the melt component contained in the active ingredient-containing layer 15 may be the same additive agents as the melt component contained in the molten component layer 13, or may be different.

In one embodiment, the core particle 10 may contain a polymer having compatibility with the melt component contained in the molten component layer 13 in the active ingredient-containing layer 15. The expression that the polymer is “compatible” with respect to the melt component means that the melt component and the polymer are not separated from each other. Alternatively, it indicates a state in which the polymer is dispersed in the melt component, or a state in which the melt component is dispersed in the polymer. In one embodiment, the state in which the melt component and the polymer are not separated can be confirmed by an increase in the viscosity of the mixture (liquid or semi-solid having fluidity) when the melt component and the polymer are mixed and the melt component is melted. By using a polymer having compatibility with the melt component, the viscosity of the surface of the molten component layer 13 is further improved, and the active ingredient-containing layer 15 can be adhered more stably. As a combination of a polymer having compatibility with the melt component, when the melt component is stearic acid or lauromacrogol, the polymer is an aminoalkyl methacrylate copolymer, an ammonioalkyl methacrylate copolymer, a methacrylic acid copolymer, hypromellose acetate succinate or polyvinylpyrrolidone can be preferably combined. More preferably, when the melt component is stearic acid, an aminoalkyl methacrylate copolymer, an ammonioalkyl methacrylate copolymer or polyvinylpyrrolidone can be combined as the polymer. Alternatively, when the melt component is lauromacrogol, an aminoalkyl methacrylate copolymer, an ammonioalkyl methacrylate copolymer, a methacrylic acid copolymer or a hypromellose acetate succinate can be preferably combined.

When the polymer is contained in the active ingredient-containing layer 15, the content of the melt component in the core particle 10 is preferably equal to or higher than the content of the polymer. For example, in the core particle 10, the blending ratio of the melt component and the polymer is preferably 20:1 to 1:1 and more preferably 4:1 to 1:1.

The active ingredient-containing layer 15 contains the active ingredient as a main component. The active ingredient contained in the active ingredient-containing layer 15 is not particularly limited, but is an active ingredient which can form the active ingredient-containing layer 15 by the melt layering method. In other words, the active ingredient contained in the active ingredient-containing layer 15 is selected from an active ingredient which is not denatured or does not show a significant increase in related substances due to contact with the melt component. The active ingredient-containing layer 15 preferably contains 50% by mass or more of the active ingredient with respect to the total mass of substances contained in the active ingredient-containing layer 15. In other words, in the case where the active ingredient-containing layer 15 contains the melt component or polymer, in the active ingredient-containing layer 15, it is preferable to contain a small amount of the melt component or polymer on the surface of the molten component layer 13 to an extent by which the active ingredient-containing layer 15 can be formed. Thereby, the content of the active ingredient in the core particle 10 can be effectively increased.

### [Method for Manufacturing Core Particle 10]

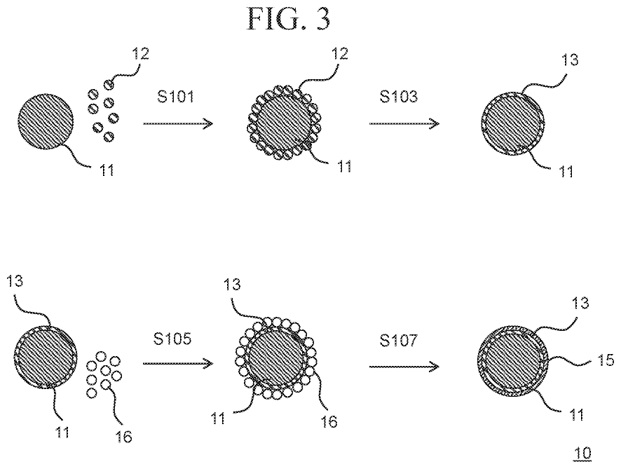

FIG. 3 is a flow diagram illustrating a method for manufacturing a core particle 10 according to one embodiment of the present invention. A core substance 11 and melt components 12 are mixed, and the melt components 12 are placed on the surface of the core substance 11 (S101). Further, the melt components 12 are melted by the melt layering method to form the molten component layer 13 on the surface of the core substance 11 (S103). At this time, the core substance 11 and the melt components 12 are heated to a temperature equal to or higher than the melting point of the melt components 12. Considering the temperature range generally used in the melt layering method, the heating temperature is 100° C. or lower. Further, when an absorbent is used as the core substance 11, it is preferable that the melt components 12 are arranged not only on the surface of the core substance 11 but also in the pores connected to the surface of the core substance 11, thereby imparting an anchor effect to the core substance 11 to the molten component layer 13 to improve the adhesion of the molten component layer 13 to the core substance 11.

The core substance 11 on which the molten component layer 13 is arranged is mixed with active ingredients 16, and the active ingredients 16 are arranged on the surface of the molten component layer 13 (S105). Further, the surface of the molten component layer 13 is melted by the melt layering method to form an active ingredient-containing layer 15 on the surface of the molten component layer 13 (S107).

In one embodiment, in the case where melt components are further contained in the active ingredient-containing layer 15 together with the active ingredients 16, the melt components contained in the active ingredient-containing layer 15 together with the active ingredients 16 are mixed with the core substance 11 on which the molten component layer 13 is arranged. In the case where an additive agent having a melting point lower than the melting point of the melt component contained in the molten component layer 13 is selected as the melt component contained in the active ingredient-containing layer 15, when the active ingredient-containing layer 15 is formed by the melt layering method, the active ingredient-containing layer 15 is formed by heating to a temperature higher than the melting point of the melt component contained in the active ingredient-containing layer 15 and lower than the melting point of the melt component contained in the molten component layer 13, thereby, the active ingredient-containing layer 15 can be formed on the surface of the molten component layer 13 without significant effect on the surface structure of the molten component layer 13 or changing the surface structure of the molten component layer 13. On the other hand, in the case where an additive agent having a melting point higher than the melting point of the melt component contained in the molten component layer 13 is selected as the melt component contained in the active ingredient-containing layer 15, when the active ingredient containing layer 15 is formed by the melt layering method by heating to a temperature higher than the melting point of the melt component contained in the active ingredient-containing layer 15, the surface of the molten component layer 13 is slightly melted, and the interface between the molten component layer 13 and the active ingredient-containing layer 15 is fused, whereby the adhesiveness of the active ingredient-containing layer 15 with respect to the molten component layer 13 can be improved. It is preferable to perform the melt layering in a temperature range in which the active ingredient 16 is not denatured or a significant increase in related substances is not observed.

In one embodiment, in the case where a polymer is further contained together with the active ingredient 16 in the active ingredient-containing layer 15, the polymer together with the active ingredient 16 is mixed with the core substance 11 on which the molten component layer 13 is arranged. A polymer having compatibility with the melt component contained in the molten component layer 13 can be used. In the embodiment, by using the polymer having compatibility with the melt component contained in the molten component layer 13, the viscosity of the surface of the molten component layer 13 is further improved, and the active ingredient-containing layer 15 can be adhered more stably.

### [Modified Example of the Core Particle]

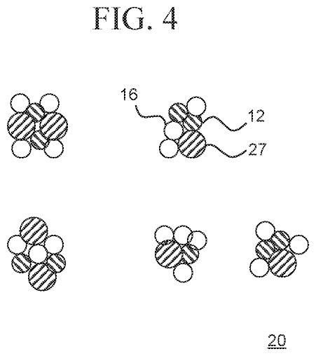

Although the above-described core particle has a structure in which the molten component layer and the active ingredient-containing layer are stacked on the surface of the core particle, the core particle of the embodiment is not limited thereto. As a modified example of the core particle, a core particle which includes no core substance will be explained. FIG. 4 is a schematic diagram (cross-sectional view) which shows a core particle 20 according to one embodiment. The core particle 20 includes an active ingredient 16, a melt component 12, and a polymer 27 according to necessity. The core particle 20 is a particle in which the active ingredient 16 and the melt component 12 are bound by the melt granulation. In the case where the polymer 27 is contained, the core particle 20 is a particle in which the active ingredient 16, the melt component 12 and the polymer 27 are bound by the melt granulation.

In the case where the polymer 27 is contained, the polymer 27 is selected from additive agents having compatibility with the melt component 12 and are applicable to melt granulation to bind the melt component 12 with the polymer 27 in the core particle 20. In the embodiment, the melt component 12 can be selected from the melt component explained in the core particle 10. In addition, the polymer 27 can be selected from the polymer explained in the core particle 10 and can be combined with the melt component 12. In the embodiment, the active ingredient is not particularly limited, but is an active ingredient which can bind with the melt component 12 and/or the polymer 27 by the melt granulation. In other words, the active ingredient 16 is selected from an active ingredient which is not denatured or does not show a significant increase in related substances due to contact with the melt component 12 and/or the polymer 27. For this reason, detailed explanations for these additive agents are omitted.

In the core particle 20, the active ingredient 16 and the melt component 12 need only form core particle, and the active ingredient 16 and the melt component 12 may be mixed with each other by melting, or may have a structure in which a part of the active ingredient 16 and the melt component 12 are bonded to each other by melting. In one embodiment, a structure is preferably in which a part of a particle of the active ingredient 16 and a part of a particle of the melt component 12 are bonded to each other by melting.

In the case where the polymer 27 is contained, in the core particle 20, the active ingredient 16, the melt component 12 and the polymer 27 need only form a core particle, and the active ingredient 16, the melt component 12 and the polymer 27 may be mixed with each other by melting, or may have a structure in which a part of the active ingredient 16, the melt component 12 and the polymer 27 are bonded to each other by melting. In one embodiment, a structure is preferably in which a part of a particle of the active ingredient 16, a part of a particle of the melt component 12 and a part of a particle of the polymer 27 are bonded to each other by melting.

The core particle 20 preferably contains the active ingredient 16 as a main component. The core particle 20 preferably contains 50% by mass or more of the active ingredient 16 with respect to the total mass of the active ingredient 16 and the melt component 12. In other words, it is preferable to contain a small amount of the melt component 12 to an extent by which core particle 20 can be formed. Thereby, the content of the active ingredient 16 in the core particle 20 can be effectively increased. In addition, uniformity of particle size in the granulated product is high in the core particle 20.

In the case where the polymer 27 is contained, the core particle 20 preferably contains 50% by mass or more of the active ingredient 16 with respect to the total mass of the active ingredient 16, the melt component 12 and the polymer 27. In other words, it is preferable to contain a small amount of the melt component 12 and the polymer 27 to an extent by which core particle 20 can be formed. Thereby, the content of the active ingredient 16 in the core particle 20 can be effectively increased. In addition, uniformity of particle size in the granulated product is high in the core particle 20.

### [Method for Manufacturing Core Particle 20]

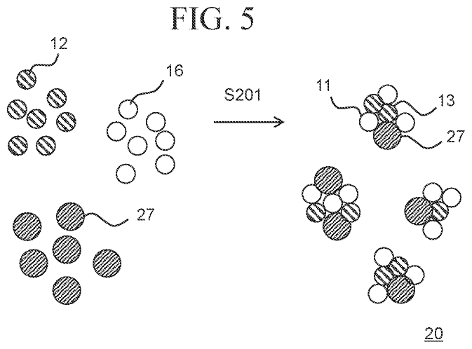

FIG. 5 is a flow diagram which illustrates a manufacturing method of core particle 20 according to one embodiment. Active ingredients 16 and melt components 12 are mixed, and the active ingredients 16 and the melt components 12 are granulated by melting to form core particles 20 (S201). At this time, these products are heated to a temperature equal to or higher than a melting point of the melt components 12. Considering the temperature range generally used in the melt granulation method, the heating temperature is 100° C. or lower. Further, it is preferable to perform the melt granulation in a temperature range in which the active ingredients 16 are not denatured or a significant increase in related substances is not observed.

When polymers 27 are contained in the core particle 20, the active ingredients 16, melt components 12 and the polymers 27 are mixed, and the active ingredients 16, melt components 12 and the polymers 27 are granulated by melting to form core particles 20 (S201). At this time, these products are heated to a temperature equal to or higher than the melting point of the melt components 12 and equal to or higher than a glass transition point of the polymers. Considering the temperature range generally used in the melt granulation method, the heating temperature is 100° C. or lower. Further, it is preferable to perform the melt granulation in a temperature range in which the active ingredients 16 are not denatured or a significant increase in related substances is not observed.

By manufacturing the core particles 20 under such temperature control, the active ingredients 16 and the melt components 12 can be bound to each other to obtain the core particles 20. Alternatively, the active ingredients 16, melt components 12 and the polymers 27 can be bound to each other to obtain the core particles 20. As described above, the core particles 20 can be easily manufactured by the melt granulation method.

### [Modified Example of the Film-Coated Granule]

The above-described core particle 10 or core particle 20 contains the melt component and can be used as the core particle of the film-coated granule according to the embodiment. Therefore, the film-coated granule can be obtained using the core particle 20 instead of the core particle 10.

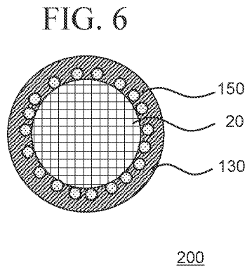

FIG. 6 is a schematic diagram (cross-sectional view) which shows a film-coated granule 200 according to one embodiment. The film-coated granule 200 includes the core particle 20 containing the melt component, porous substances 150 absorbing the plasticizer and arranged on a surface of the core particle 20, and a film 130. Also, a constitution of the film 130 may be the same as the constitution described above, and a detailed explanation is omitted.

### [Method for Manufacturing the Film-Coated Granule]

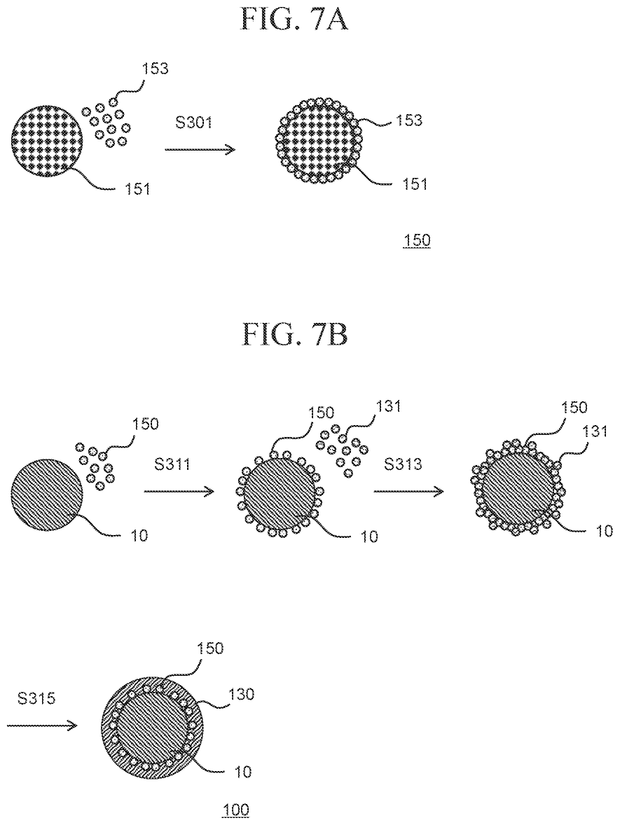

A method for manufacturing the film-coated granule will be explained. FIG. 7A is a flow diagram which illustrates a step of preparing a porous substance 150 absorbing a plasticizer according to one embodiment. Plasticizers 153 are absorbed to the porous substance 151. For example, the porous substance 151 and plasticizers 153 are mixed in a mortar to absorb the plasticizers 153 to the porous substance 151 (S301). At this time, it is preferable to absorb the plasticizers 153 not only on the surface of the porous substance 151 but also on the interior of the pores opened on the surface of the porous substance 151.

The above-described core particles are prepared, and the core substances 150 absorbing the plasticizer are absorbed to the core particles. FIG. 7B is a flow diagram which illustrates a manufacturing method of a film-coated granule 100 according to one embodiment. In FIG. 7B, an example using the core particle 10 is shown, but the embodiment is not limited thereof, and the above-described core particle 20 can be used. The porous substances 150 absorbing the plasticizer and the core particle 10 containing the melt component are mixed, and the melt component 12 is molten by the melt granulation method to obtain a particle (first particle) in which the porous substances 150 are absorbed on the surface of the core particle 10 (S311). At this time, the core particle 10 and the porous substances 150 are heated to a temperature (first temperature) equal to or higher than the melting point of the melt components 12. Considering the temperature range generally used in the melt granulation method, the heating temperature is 100° C. or lower.

Next, the core particle 10 absorbing the porous substances 150 is mixed with polymers 131, and the melt component 12 is molten by the melt granulation method to obtain a particle (second particle) in which the polymers 131 are absorbed on the surface of the core particle 10 (S313). At this time, the core particle 10 and the polymers 131 are heated to a temperature (second temperature) equal to or higher than the melting point of the melt components 12. Considering the temperature range generally used in the melt granulation method, the heating temperature is 100° C. or lower. Here, the first temperature to absorb the porous substances 150 to the core particle 10 and the second temperature to absorb the polymers 131 to the core particle 10 may be the same or different. In addition, in one embodiment, a pharmaceutically acceptable additive agent may be mixed with the core particle 10 absorbing the porous substances 150 and the polymers 131, and melt component 12 may be molten by the melt granulation method to obtain a particle (second particle) in which one or more pharmaceutically acceptable additive agents together with the polymers 131 are absorbed on the surface of the core particle 10 (S313).

The polymers 131 absorbed on the core particle 10 form a film by a curing process (S315). Thereby, a film 130 containing the porous substance 151, the plasticizers 153 and the polymers 131 can be formed on the core particle 10. At this time, the core particle 10 absorbing the polymers 131 is heated to a temperature (third temperature) equal to or higher than the second temperature. In the embodiment, because the plasticizers 153 absorbed on the porous substance 151 gradually seeps by a centrifugal force during the curing, the glass transition point of the polymers 131 can be lowered. Thereby, the minimum film forming temperature of the film 130 is lowered, and a compact film can be formed.

In one embodiment, a lubricant may be added in the curing process. A suppressing effect of aggregation in the curing process can be improved by the addition of the lubricant.

The minimum film forming temperature can be lowered by directly adding plasticizer to the polymer. However, because the polymer is rapidly softened in the melt granulation, the aggregation occurs by adhesion of the particles to each other. On the other hand, in the embodiment, because the plasticizer 153 absorbed on the porous substance 151 gradually seeps in the curing, the aggregation by rapid softening of the polymer can be suppressed while the minimum film forming temperature can be lowered. In addition, the manufacturing method of the embodiment can form the film 130 in a short time compared with the prior art.

In one embodiment, when the polymer 131 absorbed on the core particle 10 forms the film, a lubricant may be further added. The lubricant can be selected from known additive agents, for example, light anhydrous silicic acid, talc, carnauba wax, hydrous silicon dioxide, stearic acid, magnesium stearate, calcium stearate, sodium stearyl fumarate, magnesium aluminometasilicate and the like can be used. A suppressing effect of aggregation in the melt granulation can be improved by the addition of the lubricant.

In addition, in the curing process, not only the above-described dry method, but also a wet process in which granules are heated while spraying a solvent such as water in the state of fluidizing the granules by an apparatus such as a fluid bed granulator may be used.

### [Pharmaceutical Preparation]

A pharmaceutical preparation using the film-coated granules 100 or film-coated granules 200 can be manufactured. For example, the film-coated granules 100 or film-coated granules 200 and one or more pharmaceutically acceptable additive agents may be mixed to make a pharmaceutical composition. In addition, the pharmaceutical composition may be tableted to make a tablet. Further, a pharmaceutical composition to which the disintegrant is added may be tableted to obtain an orally disintegrating tablet. Furthermore, the pharmaceutical composition may be encapsulated to form a capsule.

### EXAMPLE 1

140 g of hydrated silicon dioxide (Fuji Silysia Chemical Ltd., Sylopure (registered trademark) P100) as a core substance, and 84 g of stearic acid (NOF CORPORATION, Plant) and 112 g of stearic acid (BASF Japan, Kolliwax (registered trademark) S Fine) as melt components were put into a high-speed stirring granulator (EARTHTECHNICA CO., LTD., FS-GS-2J), and the stearic acid is absorbed on the hydrated silicon dioxide at a product temperature of 80° C. for 13 minutes.

76.3 g of the obtained core substance containing the melt components, 198.3 g of sitagliptin phosphate as an active ingredient, and 25.4 g of aminoalkyl methacrylate copolymer E (Evonik, Eudragit (registered trademark) EPO) as a polymer were put into fluid bed granulator (Powrex Corporation, MP-01), and granulation was performed at a product temperature of 65° C. for 30 minutes to obtain core particles.

12 g of light anhydrous silicic acid (FREUND CORPORATION, Adsolider (registered trademark) 101) as a porous substance, 30 g of triethyl citrate (MORIMURA BROS., INC., CITROFLEX (registered trademark) 2) as a plasticizer were mixed by a mortar to absorb the triethyl citrate to the light anhydrous silicic acid.

300 g of the core particles and 42 g of the light anhydrous silicic acid absorbing the triethyl citrate were put into the fluid bed granulator (Powrex Corporation, MP-01), and granulation was performed at a product temperature of 55° C. for 10 minutes to absorb the light anhydrous silicic acid absorbing the triethyl citrate to surfaces of the core particles.

342 g of the obtained particles and 100 g of hypromellose acetate succinate (Shin-Etsu Chemical Co., Ltd., Shin-Etsu AQOAT (registered trademark) AS-LF) as a polymer were put into the fluid bed granulator (Powrex Corporation, MP-01), and granulation was performed at a product temperature of 55° C. for 10 minutes to absorb the hypromellose acetate succinate to the core particles, then particles before a curing process were obtained.

44.2 g of light anhydrous silicic acid (FREUND CORPORATION, Adsolider (registered trademark) 101) as a lubricant was further added, and curing was performed at a supply air temperature of 90° C. for 1 hour to obtain film-coated granules of Example 1.

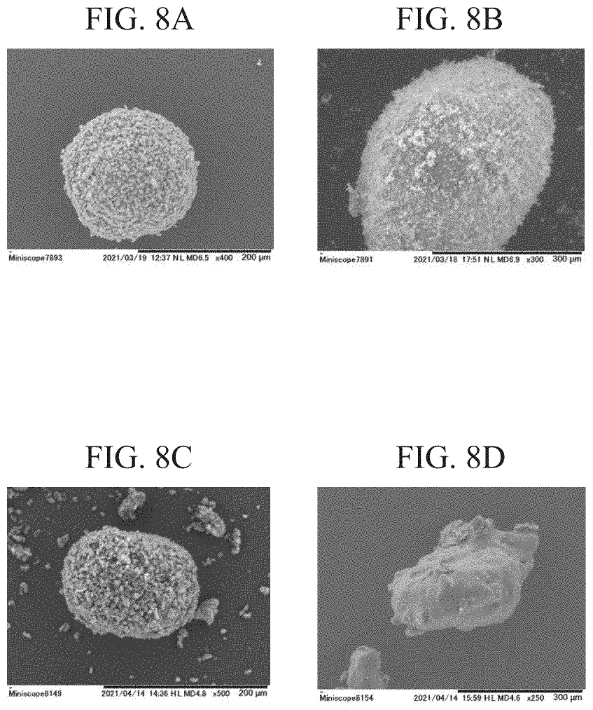

FIG. 8A is a scanning electron microscope image of a granule of Example 1 before curing, and FIG. 8B is a scanning electron microscope image of a granule of Example 1 after curing. Particles of the hypromellose acetate succinate attached on surfaces of the particles were observed in FIG. 8A, but formation of the film by curing were observed in FIG. 8B. In the example, it was shown that the film could be formed without aggregation.

### EXAMPLE 2

Film-coated granules were manufactured by changing the active ingredient of the core particle to lacosamide. 21.5 g of the lacosamide as the active ingredient, 4.0 g of ammonioalkyl methacrylate copolymer (Evonik, Eudragit (registered trademark) RSPO) as the core substance, and 2.6 g of glycerin monostearate (RIKEN Vitamin Co., Ltd., RIKEMAL (registered trademark) S-100P) as the melt component were put into a horizontal twin screw mixer with an isothermal water circulation apparatus (Caleva UK, Mixer Torque Rheometer; MTR), and granulation was performed at a product temperature of 72° C. for 30 minutes to obtain a core particles containing the melt component.

28.2 g of the core particles, and 4.0 g of the light anhydrous silicic acid absorbing the triethyl citrate prepared in the Example 1 were put into the horizontal twin screw mixer with an isothermal water circulation apparatus (Caleva UK, Mixer Torque Rheometer; MTR), and granulation was performed at a product temperature of 55° C. for 10 minutes to absorb the light anhydrous silicic acid absorbing the triethyl citrate to surfaces of the core particles.

32.2 g of the obtained particles and 9.4 g of hypromellose acetate succinate (Shin-Etsu Chemical Co., Ltd., Shin-Etsu AQOAT (registered trademark) AS-LF) as the polymer were put into the horizontal twin screw mixer with an isothermal water circulation apparatus (Caleva UK, Mixer Torque Rheometer; MTR), and granulation was performed at a product temperature of 55° C. for 10 minutes to absorb the hypromellose acetate succinate to the surface of the core particles, then particles before a curing process were obtained. After that, curing was performed at a supply air temperature of 90° C. for 1 hour to obtain film-coated granules of Example 2.

FIG. 8C is a scanning electron microscope image of a granule of Example 2 before curing process, and FIG. 8D is a scanning electron microscope image of a granule of Example 2 after curing process. Particles of the hypromellose acetate succinate attached on surfaces of the particles were observed in FIG. 8C, but formation of the film by the curing process were observed in FIG. 8D. In the example, it was shown that the film could be formed without aggregation.

### COMPARATIVE EXAMPLE 1

As Comparative example 1, the effect of containing the melt component in the core particle was examined. 300 g of microcrystalline cellulose (Asahi Kasei Corporation, Celphere (registered trademark) CP102) as the core particle, and 42 g of the light anhydrous silicic acid absorbing the triethyl citrate prepared in the Example 1 were put into fluid bed granulator (Powrex Corporation, MP-01), and granulation was performed at a product temperature of 55° C. for 10 minutes to absorb the light anhydrous silicic acid absorbing the triethyl citrate to surfaces of the core particles.

342 g of the obtained particles and 100 g of hypromellose acetate succinate (Shin-Etsu Chemical Co., Ltd., Shin-Etsu AQOAT (registered trademark) AS-LF) as the polymer were put into the fluid bed granulator (Powrex Corporation, MP-01), and granulation was performed at a product temperature of 55° C. to 65° C. for 10 minutes.

In the Comparative example 1, in the film coating process, the hypromellose acetate succinate did not absorb on the surface of the particles under the low temperature condition of 55° C. In addition, aggregations occurred under the high temperature condition of 65° C. For these reasons, no film-coated granule could be obtained in the Comparative example 1. From the results of the Comparative example 1, it was revealed that the melt component contained in the core particle is necessary to absorb the polymer and the porous substance absorbing the plasticizer to the surface of the core particle.

### COMPARATIVE EXAMPLE 2

As Comparative example 2, the effect of containing the porous substance absorbing the plasticizer was examined. 300 g of core particles prepared in the Example 1, and 100 g of hypromellose acetate succinate (Shin-Etsu Chemical Co., Ltd., Shin-Etsu AQOAT (registered trademark) AS-LF) as the polymer were put into the fluid bed granulator (Powrex Corporation, MP-01), and granulation was performed at a product temperature of 55° C. for 10 minutes to absorb the hypromellose acetate succinate to the surfaces of the core particles, then particles before a curing process were obtained. After that, curing was performed at a supply air temperature of 90° C. for 1 hour.

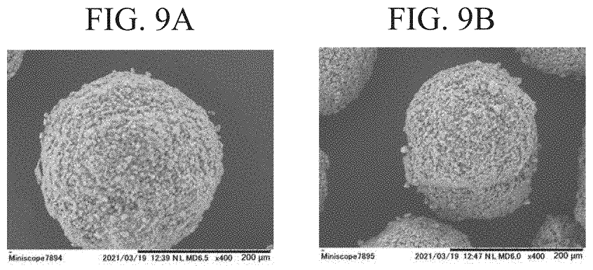

FIG. 9A is a scanning electron microscope image of a granule of Comparative example 2 before curing, and FIG. 9B is a scanning electron microscope image of a granule of Comparative example 2 after curing. From the comparison result of FIG. 9A and FIG. 9B, it was observed that the polymer absorbed on the surface of the core particles did not form any film. From the result of the Comparative example 2, it was revealed that the plasticizer absorbed on the surface of the porous substance is necessary to form the film of the polymer.

### COMPARATIVE EXAMPLE 3

As Comparative example 3, the effect of the porous substance to absorb the plasticizer was examined. 12 g of non-porous light anhydrous silicic acid (NIPPON AEROSIL CO., LTD., AEROSIL (registered trademark) 200), and 30 g of triethyl citrate (MORIMURA BROS., INC., CITROFLEX (registered trademark) 2) as a plasticizer were mixed by a mortar to absorb the triethyl citrate to the light anhydrous silicic acid.

300 g of core particles prepared in the Example 1, and 42 g of the light anhydrous silicic acid absorbing the triethyl citrate of the Comparative example 3 were put into the fluid bed granulator (Powrex Corporation, MP-01), and granulation was performed at a product temperature of 55° C. for 10 minutes to absorb the light anhydrous silicic acid absorbing the triethyl citrate to the surfaces of the core particles.

342 g of the obtained particles and 100 g of hypromellose acetate succinate (Shin-Etsu Chemical Co., Ltd., Shin-Etsu AQOAT (registered trademark) AS-LF) as a polymer were put into the fluid bed granulator (Powrex Corporation, MP-01), and granulation was performed at a product temperature of 55° C. for 10 minutes to absorb the hypromellose acetate succinate to the core particles, then particles before a curing process were obtained. After that, curing was performed at a supply air temperature of 90° C. for 1 hour.

In the Comparative example 3, in the film coating process, aggregations occurred and no film-coated granule could be obtained. From the results of the Comparative example 3, it was assumed that because the non-porous light anhydrous silicic acid did not absorb the plasticizer, the polymer reacted with the plasticizer at once when the polymer was added, then the aggregation occurred due to rapid softening of the polymer. From the results of the Comparative example 3, it was revealed that the porous substance is necessary to absorb the plasticizer.

### [Examination of Suppressing Effect of Bitter Taste]

With respect to the granules of the Example 1 and Comparative example 2, and the core particles of the Example 1, it was examined whether the bitter taste of sitagliptin phosphate can be suppressed by a sensory inspection. Five inspectors held 50 mg of the granules of the Example 1 and Comparative example 2 in their oral cavity for 20 seconds, and examined the existence or non-existence of the bitter taste. With respect to the film-coated granules of Example 1, four inspectors did not feel the bitter taste, and one inspector felt slight bitter taste. On the other hand, with respect to the granules of the Comparative example 2 in which the polymer did not form the film and the core particles of the Example 1, five inspectors felt a strong bitter taste. From these results, it was confirmed that a compact film capable of suppressing the bitter taste was formed in the film-coated granules of Example 1.

### Example 3

430.5 g of hydrated silicon dioxide (Fuji Silysia Chemical Ltd.) as a core substance, and 336.0 g of stearic acid (Kao Corporation, stearic acid 70) as melt components were put into a high-speed stirring granulator (EARTHTECHNICA CO., LTD., FS-GS-5J), and the stearic acid was absorbed on the hydrated silicon dioxide at 75° C. of a product temperature for 17 minutes.

186.15 g of the obtained core substance (absorbed particle) containing the melt components, 286.45 g of duloxetine hydrochloride as an active ingredient, and 47.60 g of aminoalkyl methacrylate copolymer E (Evonik, Eudragit (registered trademark) EPO) as a polymer were put into the high-speed stirring granulator (EARTHTECHNICA CO., LTD., FS-GS-5J), and granulation was performed at a product temperature of 65° C. for 28 minutes to obtain core particles.

520.2 g of the obtained core particles, and 15.3 g of talc (FUJI TALC INDUSTRIAL CO., LTD, ML115) were put into the high-speed stirring granulator (EARTHTECHNICA CO., LTD., FS-GS-5J), and a non-stick process was performed at a product temperature of 65° C. for 1 minute.

24 g of light anhydrous silicic acid (FREUND CORPORATION, Adsolider (registered trademark) 101) as a porous substance, 60 g of triethyl citrate (MORIMURA BROS., INC., CITROFLEX (registered trademark) 2) as a plasticizer were mixed by a mortar to absorb the triethyl citrate to the light anhydrous silicic acid.

126 g of the core particles and 25.2 g of the light anhydrous silicic acid absorbing the triethyl citrate were put into the fluid bed granulator (Powrex Corporation, MP-01), and granulation was performed at a product temperature of 65.3° C. for 5 minutes to absorb the light anhydrous silicic acid absorbing the triethyl citrate to surfaces of the core particles.

151.2 g of the obtained particles, 120 g of hypromellose acetate succinate (Shin-Etsu Chemical Co., Ltd., Shin-Etsu AQOAT (registered trademark) AS-LF) as the polymer, and 2 g of talc (FUJI TALC INDUSTRIAL CO.,LTD, ML115) as the lubricant were put into the fluid bed granulator (Powrex Corporation, MP-01), and granulation was performed at a product temperature of 65° C. for 15 minutes to absorb the hypromellose acetate succinate to the surfaces of the core particles, then particles before a curing process were obtained.

Curing was performed at a supply air temperature of 90° C. for 90 minutes to obtain film-coated granules of Example 3.

### COMPARATIVE EXAMPLE 4

126 g of the core particles of the Example 3, 120 g of hypromellose acetate succinate (Shin-Etsu Chemical Co., Ltd., Shin-Etsu AQOAT (registered trademark) AS-LF) as the polymer, and 2 g of talc (FUJI TALC INDUSTRIAL CO.,LTD, ML115) as the lubricant were put into the fluid bed granulator (Powrex Corporation, MP-01), and granulation was performed at a product temperature of 65° C. for 15 minutes to absorb the hypromellose acetate succinate to the surfaces of the core particles, then particles before a curing process were obtained.

Curing was performed at a supply air temperature of 90° C. for 90 minutes to obtain film-coated granules of Comparative example 4.

### [Evaluation of Film]

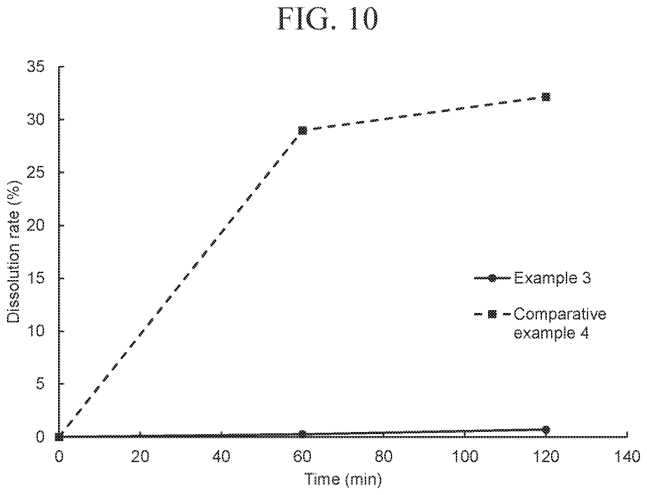

With respect to the film-coated granules of the Example 3 and the Comparative example 4, dissolution behaviors of the duloxetine hydrochloride were evaluated in accordance with a dissolution test (paddle method) of the Japanese Pharmacopoeia 17^{th} edition. Preparations for the test were prepared by filling 136.6 g of the film-coated granules of the Example 3 and 124 g of the film-coated granules of the Comparative example 4 each in hypromellose capsules. 900 ml of 1^{st} fluid for dissolution test (JP1) was used as a test solution. The rotation speed of a paddle was set to 50 rpm. The dissolution rates of duloxetine hydrochloride at 60 minutes and 120 minutes after the start of the test were measured by high performance liquid chromatography (HPLC). The dissolution rates of duloxetine hydrochloride are shown in FIG. 10. In the film-coated granules of the Example 3 which contains the porous substance absorbing the plasticizer, dissolution of the duloxetine hydrochloride was suppressed at 120 minutes after the start of the test by the coating of the hypromellose acetate succinate which is an enteric polymer. On the other hand, in the film-coated granules of the Comparative example 4 in which the enteric polymer was directly coated on the core particle, the duloxetine hydrochloride was significantly eluted compared with the Example 3. From these results, it was revealed that a compact film was formed in the Example 3 which contains the porous substance absorbing the plasticizer, thereby dissolution of the duloxetine hydrochloride was suppressed.

According to one embodiment of the present invention, film-coated granules which have a new film constitution is provided. Alternately, according to one embodiment of the present invention, a pharmaceutical preparation containing the film-coated granules which have a new film constitution is provided. Alternately, according to one embodiment of the present invention, a new dry manufacturing method of the film-coated granules is provided. Alternately, according to one embodiment of the present invention, a new dry manufacturing method of the pharmaceutical preparation containing the film-coated granules is provided.

What is claimed is:

1. A film-coated granule comprising:
a core particle having a melt component; and
a film arranged on a surface of the core particle,
wherein the film includes a porous substance, a plasticizer and a polymer.

2. The film-coated granule according to claim 1, wherein the plasticizer is selected from plasticizers arrangeable between molecules of the polymer.

3. The film-coated granule according to claim 1, wherein the plasticizer is selected from a group consisting of triethyl citrate, polyethylene glycol, propylene glycol, triacetin and tributyl acetylcitrate, and
the polymer is selected from a group consisting of hypromellose acetate succinate, hydroxypropyl cellulose, ethyl cellulose, vinyl acetate, methacrylic acid copolymer L, ammonioalkyl methacrylate copolymer and methacrylic acid copolymer S.

4. The film-coated granule according to claim 1, wherein the core particle includes a core substance, a molten component layer arranged on a surface of the core substance, and an active ingredient-containing layer arranged on a surface of the molten component layer.

5. The film-coated granule according to claim 1, wherein the core particle includes an active ingredient and the melt component, and
the active ingredient and the melt component are bound.

6. The film-coated granule according to claim 5, wherein the core particle further includes a polymer, and
the active ingredient, the melt component and the polymer are bound.

7. The film-coated granule according to claim 1, wherein the film further includes one or more first pharmaceutically acceptable additive agents.

8. A pharmaceutical preparation comprising:
the film-coated granule according to claim 1; and
one or more second pharmaceutically acceptable additive agents.

9. A manufacturing method of a film-coated granule comprising:
adsorbing a plasticizer to a porous substance;
adsorbing the porous substance absorbed with the plasticizer to a core particle having a melt component to obtain a first particle; and
adsorbing a polymer to the first particle to form a film including the porous substance, the plasticizer, and the polymer on the core particle.

10. The manufacturing method of the film-coated granule according to claim 9, wherein the polymer is adsorbed to the first particle at a first temperature, and
the film is formed on the core particle at a second temperature equal to or higher than the first temperature.

11. The manufacturing method of the film-coated granule according to claim 9, wherein the plasticizer is selected from plasticizers arrangeable between molecules of the polymer.

12. The manufacturing method of the film-coated granule according to claim 9, wherein the plasticizer is selected from a group consisting of triethyl citrate, polyethylene glycol, propylene glycol, triacetin and tributyl acetylcitrate, and
the polymer is selected from a group consisting of hypromellose acetate succinate, hydroxypropyl cellulose, ethyl cellulose, vinyl acetate, methacrylic acid copolymer L, ammonioalkyl methacrylate copolymer and methacrylic acid copolymer S.

13. The manufacturing method of the film-coated granule according to claim 9, wherein the melt component is absorbed to a core substance to form a molten component layer,
an active ingredient is absorbed to the melt component, and
an active ingredient-containing layer including the melt component and the active ingredient is formed to obtain the core particle.

14. The manufacturing method of the film-coated granule according to claim 9, wherein the melt component and an active ingredient are bound, and
the core particle including the melt component and the active ingredient is obtained.

15. The manufacturing method of the film-coated granule according to claim 9, wherein the melt component, an active ingredient and a polymer are bound, and
the core particle including the melt component, the active ingredient and the polymer is obtained.

16. The manufacturing method of the film-coated granule according to claim 9, wherein one or more pharmaceutically acceptable lubricants is further added when forming the film on the core particle.

17. A manufacturing method of a pharmaceutical preparation comprising:
mixing the film-coated granule obtained by the manufacturing method of the film-coated granule according to claim 9 and one or more second pharmaceutically acceptable additive agents to obtain a mixture; and
tableting the mixture.
